(19) United States
(12) Patent Application Publication
(10) Pub. No.: US 2014/0212513 A1
(43) Pub. Date: Jul. 31, 2014

(54) TOPICAL COMPOSITIONS COMPRISING DEAD SEA WATER AND USES THEREOF

(71) Applicant: AHAVA - DEAD SEA LABORATORIES LTD., Airport City (IL)

(72) Inventors: Isabelle Rachel AFRIAT-STALOFF, Ma'ale Adumim (IL); Meital Portugal COHEN, Jerusalem (IL); Zeevi MAOR, Kibbutz Kalia (IL); Dror COHEN, D.N the Dead Sea (IL)

(73) Assignee: AHAVA - DEAD SEA LABORATORIES LTD., Airport City (IL)

(21) Appl. No.: 14/167,421

(22) Filed: Jan. 29, 2014

Related U.S. Application Data
(60) Provisional application No. 61/758,057, filed on Jan. 29, 2013.

(51) Int. Cl. A61K 35/08; A61Q 19/00; A61K 8/96

(52) U.S. Cl. CPC A61K 35/08; A61K 8/965; A61Q 19/00
USPC 424/678

(57) ABSTRACT

The present disclosure provides method of inducing secretion of beta-endorphin in a skin region of a subject, the method comprises topically applying a composition comprising at least 0.5% Dead Sea Water onto a skin region of a subject. The disclosure further provides a method of modifying at least one condition of a skin region of a subject, the method comprises topically applying a composition comprising at least 0.5% Dead Sea Water onto the skin region of the subject, wherein the at least one skin condition is selected from the group consisting of mechanical and physiological skin condition and wherein the modifying of the at least one skin condition is associated with induction of skin beta-endorphin secretion in the skin region.

### FIELD OF THE INVENTION

This invention relates to topical compositions comprising Dead Sea Water and uses thereof.

### BACKGROUND OF THE INVENTION

Beta-endorphin (β-endorphin) is a 31 amino acids long peptide known to be produced and released by nerve cells (neurones), thus it is also referred to as a neuropeptide. Beta-endorphin is produced from the larger precursor pro-opiomelanocortin (POMC) as follows: under the effect of specific serine-proteinase type enzymes present in the hypothalamus, the polypeptide chain of POMC is split into small peptides, particularly into N-terminal peptide (111 amino acids), adenotropic hormone (ACTH, 39 amino acids) and beta-lipotropin (beta-LPH, 91 amino acids). ACTH is split into the hormone responsible for stimulating melanocytes (alpha-MSH) and corticotropin-like intermediate peptide (CLIP), while beta-LPH is split into gamma-LPH (59 amino acids) and beta-endorphin (31 amino acids).

The amino acid sequence of β-endorphin is the following sequence: Tyr-Gly-Gly-Phe-Met-Thr-Ser-Glu-Lys-Ser-Gln-Thr-Pro-Leu-Val-Thr-Leu-Phe-Lys-Asn-Ala-Ile-Ile-Lys-Asn-Ala-Tyr-Lys-Lys-Gly-Glu.

Beta-endorphin is also produced by cells other than neurones, particularly by skin cells and more specifically by epidermal keratinocytes and by human dermal fibroblasts. The release of beta-endorphins by the skin cells is known to be stimulated by outside stress, particularly by low-intensity ultraviolet radiation. Recently, it has been demonstrated that beta-endorphins production is also stimulated by topical application of plant extracts.

Beta-endorphins are known to play a role in controlling stress and have a number of other benefits such as slowing the growth of cancer cells, regulating of skin immune function, promoting feeling of well-being, maintaining and improving and/or healing associated with various skin conditions such as atopic dermatitis and psoriasis. Improvement in quality of life and disease coping in patients with vitiligo after group climatotherapy at the Dead Sea has been recently demonstrated [1].

Dead Sea water, salts, minerals and mud are well known for their therapeutic efficacy in treating a variety of skin conditions as well as for their cosmetic benefits [2-5].

### REFERENCES

[1] Christian Krüger et al., Acta. Derm. Venereol. 2011, 91: 152-159 “Significant Immediate and Long-term Improvement in Quality of Life and Disease Coping in Patients with Vitiligo after Group Climatotherapy at the Dead Sea”.
[2] Nissen J. B., et. al., British Journal of Dermatology 1998, 139: 1012-1019. “Increased levels of enkephalin following natural sunlight(combined with salt water bathing at the Dead Sea) and ultraviolet A irradiation”.
[3] Sukenik S., et al., J Rheumatol 1994, 21:1305-1309. “Treatment of psoriatic arthritis at the Dead Sea”.
[4] S. Halevy., et al. Journal of the European Academy of Dermatology and Venereology, 1997, 9, 237-242 “Dead Sea bath salt for the treatment of psoriasis vulgaris: a double-blind controlled study”.
[5] Maor Z. and Yehuda S. International Journal of Cosmetic Science, 1997, 19, 105-110. “Skin smoothing effects of Dead Sea minerals: comparative profilometric evaluation of skin surface”.

### SUMMARY OF THE INVENTION

The inventors of the present disclosure have surprisingly found that topical application of minerals on the skin stimulates the productions of skin beta-endorphin neuropeptides. Specifically, the inventors have studied the effect of Dead Sea Water on secretion of skin beta-endorphins and found that topical application of compositions comprising at least 0.5% Dead Sea Water on skin explants stimulates the release of skin beta-endorphins. The release of skin beta-endorphins occurred over a period of 48 hours post topical application of the compositions of the present disclosure onto a skin region. The endorphin secretion induction effect of the compositions according to the present disclosure may be utilized to modify various skin conditions, attributing the compositions both cosmetic and therapeutic benefits.

Thus, the present invention provides in one of its aspects a topical composition comprising at least 0.5% Dead Sea Water for modifying at least one condition of the skin selected from the group consisting of mechanical and physiological condition of the skin.

In another one of its aspects the present invention provides a topical composition comprising at least 0.5% Dead Sea Water for modifying at least one condition of the skin selected from the group consisting of mechanical and physiological condition of the skin, wherein the modification is associated with induction (or enhancement) of skin beta-endorphin/s secretion.

In yet another one of its aspects the present invention provides a topical composition comprising at least 0.5% Dead Sea Water for modifying at least one condition of the skin selected from the group consisting of mechanical and physiological condition of the skin, wherein the modification is associated with induction (or enhancement) of skin beta-endorphin/s secretion and wherein the secretion is induced (or enhanced) over a period of 48 hours post topical application of the composition onto a skin region of a subject.

In another one of its aspects the present invention provides a topical composition comprising at least 0.5% Dead Sea Water for inducing (or enhancing) secretion of skin beta-endorphin/s.

In a further one of it aspects the present invention provides a topical composition comprising at least 0.5% Dead Sea Water for inducing (or enhancing) secretion of skin beta-endorphin/s, wherein the secretion is induced (or enhanced) over a period of 48 hours post topical application of the composition onto a skin region of a subject.

Yet, in a further one of its aspects the present invention provides a method of modifying at least one condition of a skin region of a subject, the method comprising topically applying a composition comprising at least 0.5% Dead Sea Water onto the skin region of the subject, wherein the at least one skin condition is selected from the group consisting of mechanical and physiological skin condition and wherein the modifying of the at least one skin condition is associated with induction (or enhancement) of skin beta-endorphin secretion in the skin region.

In a further one of its aspects the present invention provides a method of modifying at least one condition of a skin region of a subject, the method comprising topically applying a composition comprising at least 0.5% Dead Sea Water onto the skin region of the subject, wherein the at least one skin condition is selected from the group consisting of mechanical and physiological skin condition and wherein the modifying of the at least one skin condition is associated with induction (or enhancement) of skin beta-endorphin secretion in the skin region, wherein the secretion is induced (or enhanced) over a period of 48 hours post the topical application of the composition onto the skin region of the subject.

In yet a further one of its aspects the present invention provides a method of inducing (or enhancing) the secretion of beta-endorphin is a skin region of a subject, the method comprising topically applying a composition comprising at least 0.5% Dead Sea Water onto the skin region of the subject.

In a further one of its aspects the present invention provides a method of inducing (or enhancing) the secretion of beta-endorphin is a skin region of a subject, the method comprising topically applying a composition comprising at least 0.5% Dead Sea Water onto the skin region of the subject, wherein the secretion is induced (or enhanced) over a period of 48 hours post the topical application of the composition onto the skin region of the subject.

Yet, in a further one of its aspects the present invention provides use of at least 0.5% Dead Sea Water in the manufacture of a topical composition for modifying at least one condition of the skin selected from the group consisting of mechanical and physiological condition of the skin.

In another one of its aspects the present invention provides use of at least 0.5% Dead Sea Water in the manufacture of a topical composition for modifying at least one condition of the skin selected from the group consisting of mechanical and physiological condition of the skin, wherein the modification is associated with induction (or enhancement) of skin beta-endorphin/s secretion.

In yet another one of its aspects the present invention provides use of at least 0.5% Dead Sea Water in the manufacture of a topical composition for modifying at least one condition of the skin selected from the group consisting of mechanical and physiological condition of the skin, wherein the modification is associated with induction (or enhancement) of skin beta-endorphin/s secretion and wherein the secretion is induced (or enhanced) over a period of 48 hours post topical application of the composition onto a skin region of a subject.

In another one of its aspects the present invention provides use of at least 0.5% Dead Sea Water in the manufacture of a topical composition for inducing (or enhancing) of skin beta-endorphin/s secretion.

Yet, in another one of its aspects the present invention provides use of at least 0.5% Dead Sea Water in the manufacture of a topical composition for inducing (or enhancing) of skin beta-endoiphin/s secretion, wherein the secretion is induced (or enhanced) over a period of 48 hours post topical application of the composition onto a skin region of a subject.

In a further one of its aspects the present invention provides compositions according to the invention formulated as a personal skin care product, a dermatological formulation or as a skin care formulation.

### BRIEF DESCRIPTION OF THE DRAWINGS

In order to understand the invention and to see how it may be carried out in practice, embodiments will now be described, by way of non-limiting example only, with reference to the accompanying drawings, in which:

FIGS. 1A-1B provide schematic illustration of the effect of Dead Sea Water on skin beta-endorphins secretion observed 24 hours and 48 hours following topical application.

FIG. 2 provides schematic illustration of the effect of Dead Sea Water on skin beta-endorphins secretion observed 48 hours following topical application, the data is normalized to cell viability.

### DETAILED DESCRIPTION OF THE INVENTION

The present invention provides in one of its aspects a topical composition comprising at least 0.5% Dead Sea Water for modifying at least one condition of the skin selected from the group consisting of mechanical and physiological condition of the skin. In some embodiments the modification is associated with induction of skin beta-endorphin secretion.

In yet another one of its aspects the present invention provides a topical composition comprising at least 0.5% Dead Sea Water for modifying at least one condition of the skin selected from the group consisting of mechanical and physiological condition of the skin, wherein the modifying is associated with induction of skin beta-endorphin secretion.

As used herein the term “Dead Sea water” (herein abbreviated DSW) refers to the saline waters obtained from the Dead Sea (Israel) region or an aqueous solution prepared by dissolving Dead Sea minerals in an aqueous medium. The term also encompasses aqueous solutions which simulate such natural solution, namely having at least one parameter substantially identical to that measured for the natural DSW, said parameter being at least one of salt content, salt concentration, concentration of a particular cation or anion, ratio of divalent cations to monovalent cations, TDS (Total Dissolved Salt, w/v), soluble natural substances, and other parameters known to define or characterize natural DSW.

In some embodiments, the Dead Sea water having:

1. a specific density of 1.25-1.35 g/ml,

2. pH=4.6-5.6 (at 25° C.), and/or

3. less than 100 cfu/g of non-pathogenic microbes.

In some further embodiments, the DSW is a clear colorless viscous liquid (at 25° C.).

The Dead Sea water having the above physical characteristics is a concentrated extract of Dead Sea Water comprising (among other metal salt ions) Ca⁺², Mg⁺², Na⁺ and K⁺ and high concentrations of anions such as Cl⁻ and Br⁻.

In some embodiments, the concentrations of these ions are, as assessed by a water analysis carried out by the Geological Survey of Israel:
Calcium (Ca⁺²): 35,000-40,000 mg/L
Chloride (Cl⁻): 320,000-370,000 mg/L
Magnesium (Mg⁺²): 92,000-95,000 mg/L
Sodium (Na⁺): 1800-3200 mg/L
Potassium (K⁺): 2,500 mg/L, and
Bromide (Br⁻): 10,000-12,000 mg/L.
   Other minerals may also exist in the waters.

In some embodiments, the Dead Sea Water comprises:
Calcium (Ca⁺²): 35,000-40,000 mg/L
Chloride (Cl⁻): 320,000-370,000 mg/L
Magnesium (Mg⁺²): 92,000-95,000 mg/L
Sodium (Na⁺): 2400-3200 mg/L
Potassium (K⁺): 2,500 mg/L, and
Bromide (Br⁻): 10,000-12,000 mg/L.
   Other minerals may also exist in the waters.

In other embodiments, the Dead Sea Water comprises:
Calcium (Ca⁺²): 5,000-10,000 mg/L
Chloride (Cl⁻): 315,000-360,000 mg/L
Magnesium (Mg⁺²): 100,000-150,000 mg/L
Sodium (Na⁺): 1800-2200 mg/L
Potassium (K⁺): 1,000-2,000 mg/L, and
Bromide (Br⁻): 5,000-10,000 mg/L.
   Other minerals may also exist in the waters.

In some further embodiments, the Dead Sea Water comprises:
Calcium (Ca⁺²) 34,000-40,000 mg/L
Chloride (Cl⁻) 320,000-370,000 mg/L
Magnesium (Mg⁺²) 90,000-95,000 mg/L
Potassium (K⁺) 1,300-2,200 mg/L
Sodium (Na⁺) 1,500-2,800 mg/L
Bromide (Br⁻) 11,000-15,000 mg/L.
   Other minerals may also exist in the waters.

In some embodiments, the DSW is natural DSW which has undergone pre-treatment, e.g., having been concentrated by allowing water to evaporate, for example through solar evaporation, thereafter reconstituted to afford a solution (such as the commercially available “Maris Sal”, or “Maris Aqua” AHAVA, Israel, Source: Geological Survey—Ministry of National Infrastructures, State of Israel, especially for Ahava-Dead Sea Laboratories). This solution has an overall salt concentration (constituting the original salt composition) of 15, 20, 25, 30, 35, 40 or 45% or intermediate or greater concentrations.

In some embodiments, the DSW has an overall salt concentration of 40%.

In some embodiments the Dead Sea Water may constitute at least 0.5% of the total weight of the composition of the invention and up to 100% of the total weight of the composition of the invention. In some embodiments the Dead Sea Water may constitute about 0.5% of the total weight of the composition of the invention, at times 0.75%, 0.8%, 1.0%, 2.0% or 2.5% Dead Sea Water. In some embodiments the Dead Sea Water may constitute about 5.0, 5.5, 6.0, 6.5, 7.0, 7.5, 8.0, 8.5, 9.0, 9.5, 10, 15, 20, 25, 30, 35, 40, 45, 50, 55, 60, 65, 70, 75, 80, 85, 90 and 95% of the total weight of the composition. In some embodiments the composition is DSW per se i.e., 100% DSW.

In some embodiments the composition is formulated as a leave on composition e.g., a leave on cream. To this end, the Dead Sea Water may constitute about 2.4% of the total weight of the composition of the invention.

In some embodiments the composition is a rinse-off composition. To this end, the Dead Sea Water may constitute at least 0.5% and up to 100% of the total weight of the composition of the invention.

As used herein, a “leave on” (in contrary to “rinse off”) composition/formulation refers to a composition/formulation that may be in prolonged contact with the skin and can be applied to a skin region without the need to remove it from the skin (e.g., by wiping or rinsing it off) in any way.

In some embodiments, the leave-on composition/formulation may be adapted to be applied to a skin region and to be left on the skin for a time sufficient to modify at least one mechanical and/or at least one physiological condition of the skin.

In some embodiments the composition/formulation is a facial formulation.

The term “topical” as used herein refers to the application of a composition according to the invention directly onto at least a portion/region of a subject's skin (human's or non-human's skin) so as to achieve a desired effect e.g., modifying at least one mechanical and/or at least one physiological condition of the skin, which may be a cosmetic or a therapeutic effect, at the site of application. In some embodiments, the desired effect is achieved at the site of application without inducing one or more systemic effects. In some implementations of the present disclosure, a topical effect may be accompanied at least to a certain degree with at least one systemic effect which may directly affect the induction of skin beta-endorphin/s secretion. Therefore, in some embodiments, the induced secretion of skin beta-endorphins may be associated with induction of one or more systemic effects together with a local topical effect.

The term “skin” as used herein refers to any part of the human or animal skin, including the whole surface thereof, hair and nails. In the content of the present disclosure, when the compositions are topically applied onto a skin of a subject it is to be understood that the application is onto at least a portion/region of the skin. In some embodiments the skin is at least a region of facial skin.

The compositions according to the present disclosure are utilized for modifying at least one skin condition selected from the group consisting of mechanical and physiological condition of the skin. The term “modify” or any lingual variations thereof refers to changing at least one skin condition. For example, modifying may be envisages as reducing the severity of an unfavorable skin condition or improving a favorable condition of the skin. At times the condition of the skin may reflect a disease or a cosmetic condition. To this end, and as will be elaborated herein below, the term “modify” may be associated with at least one therapeutic effect and/or at least one cosmetic effect.

As used herein, the term “mechanical” condition of the skin is in line with the common knowledge. Non-limiting examples of mechanical conditions of the skin are: skin extensibility, skin defromablity, skin elasticity, skin firmness, skin suppleness etc. At times the conditions are age related conditions associated for example with wrinkles, dull skin and the like.

As used herein, the term “physiological’ condition of the skin is in line with the common knowledge. Non-limiting examples of physiological conditions of the skin are: hydration, moisture, dryness, pH, temperature, structure, thickness etc. Conditions such as skin lipid content, skin ceramide content, skin differentiation, epidermal renewal, skin cellular communication and conditions which are associated with the skin immune function are also considered physiological skin conditions. At times the conditions are age related conditions associated for example with dull skin, dry skin and the like.

In another one of its aspects the present invention provides a topical composition comprising at least 0.5% Dead Sea Water for inducing skin beta-endorphin secretion.

As used herein the terms “induction”, “promotion”, “stimulation” or any lingual variations thereof are interchangeable and refer to initiation, enhancement, modulation or increasing of the expression of beta-endorphin in the skin, thereby resulting with beta-endorphin secretion in the skin cells. To this end the terms “secretion”, “production”, “release” or any lingual variations thereof are interchangeable.

In some embodiments of the invention the secretion may be induced over a period of time of few hours e.g., 1 hour, 2 hours, 5 hours, 10 hours, 15 hours, 20 hours after topical application of the composition onto a skin region of a subject. In some embodiments the secretion may be induces over a period of at least 24 hours post topical application of the composition onto a skin region of a subject. In some further embodiments the secretion may be induced over a period of at least 48 hours post topical application of the composition onto a skin region of a subject.

In some embodiments of the invention the induction (or enhancement) of skin beta-endorphin secretion has a Tₘₐₓ of 48 hours i.e., the secretion is induced (or enhanced) for a maximum period of time of 48 hours post topical application of the composition onto a skin region of a subject.

Accordingly, in a further one of its aspects the present invention provides a topical composition comprising at least 0.5% Dead Sea Water for modifying at least one condition of the skin selected from the group consisting of mechanical and physiological condition of the skin, wherein the modification is associated with induction (or enhancement) of skin beta-endorphin/s secretion and wherein the secretion is induced (or enhanced) over a period of time of 48 hours post topical application of the composition onto a skin region of a subject.

In another one of its aspects the present invention provides a topical composition comprising at least 0.5% Dead Sea Water for inducing secretion of skin beta-endorphin/s.

In a further one of its aspects the present invention provides a topical composition comprising at least 0.5% Dead Sea Water for inducing secretion of skin beta-endorphin/s, wherein the secretion is induced over a period of 48 hours post topical application of the composition onto a skin region of a subject.

In a further one of its aspects the present invention provides a method of inducing (or enhancing) the secretion of beta-endorphin is a skin of a subject, the method comprising topical application of a composition comprising at least 0.5% Dead Sea Water onto the skin of the subject.

In a further one of its aspects the present invention provides a method of inducing (or enhancing) the secretion of beta-endorphin is a skin region of a subject, the method comprising topically applying a composition comprising at least 0.5% Dead Sea Water onto the skin region of the subject, wherein the secretion is induced over a period of 48 hours post the topical application of the composition onto the skin region of the subject.

Yet, in a further one of its aspects the present invention provides a method of modifying at least one condition of a skin region of a subject, the method comprising topically applying a composition comprising at least 0.5% Dead Sea Water onto the skin region of the subject, wherein the at least one skin condition is selected from the group consisting of mechanical and physiological skin condition and wherein the modifying of the at least one skin condition is associated with induction of skin beta-endorphin secretion in the skin region.

In a further one of its aspects the present invention provides a method of modifying at least one condition of a skin region of a subject, the method comprising topically applying a composition comprising at least 0.5% Dead Sea Water onto the skin region of the subject, wherein the at least one skin condition is selected from the group consisting of mechanical and physiological skin condition and wherein the modifying of the at least one skin condition is associated with induction of skin beta-endorphin secretion in the skin region, wherein the secretion is induced over a period of 48 hours post the topical application of the composition onto the skin region of the subject.

In some embodiments of the invention, the modification of at least one mechanical and/or physiological condition of a skin may be associated with at least one of protecting and/or improving and/or rejuvenating the state of the skin, preventing and/or treating imperfections of the skin of a subject and/or treating or preventing at least one disease or disorder of the skin.

In some embodiments of the invention, the modification of at least one mechanical and/or physiological condition of a skin may be associated with the skin immune system. Accordingly, in some embodiments of the invention the modification may be associated with immune function of the skin. In some embodiments it may be associated with reactions which are related to the skin immune system.

In some embodiments of the invention the induction (or enhancement) of skin beta-endorphin secretion may be accompanied by at least one cosmetic and/or therapeutic effect as disclosed herein.

Non-limiting examples of skin disease or disorder include dermatological inflammation; different acne types such as acne vulgaris, cystic acne, acne rosacea, acne keloidalis nuchae, acne conglobata, acne cosmetica, acne fulminans, acne medicamentosa, baby acne and Chloracne; various kinds of dermatitis; different infections such as bacterial skin infections, fungal and yeast skin infections, viral skin infections, parasitic skin infections; pruritis; cellulites; acute lymphangitis; lymphadenitis; erysipelas; cutaneous abscesses; necrotizing subcutaneous infections; scalded skin syndrome; folliculitis; furuncles; hidradenitis suppurativa; carbuncles; paronychial infections; rashes; erythrasma; impetigo; warts; molluscum contagiosum; trauma or injury to the skin (wounds); post-operative or post-surgical skin conditions; pediculosis; creeping eruption; eczemas; different types of psoriasis; pityriasis rosea; lichen planus; pityriasis rubra pilaris; edematous; erythema multiforme; erythema nodosum; grannuloma annulare; epidermal necrolysis; sunburn; photosensitivity; pemphigus; bullous pemphigoid; dermatitis herpetiformis; keratosis pilaris; callouses; corns; ichthyosis; skin ulcers; ischemic necrosis; miliaria; hyperhidrosis; moles; poison ivy; poison oak; contact dermatitis; atopic dermatitis; rosacea; purpura; moniliasis; candidiasis; baldness; alopecia; Behcet's syndrome; cholesteatoma; Dercum disease; ectodermal dysplasia; gustatory sweating; nail patella syndrome; lupus; hives; hair loss; Hailey-Hailey disease; chemical or thermal skin burns; scleroderma; aging skin; wrinkles; sun spots; necrotizing fasciitis; necrotizing myositis; gangrene; scarring; athlete's foot; ringworm and vitiligo.

Further non-limiting examples of the effects and the skin conditions which may be affected by the compositions according to the invention and which may be associated with skin beta-endorphin production are one or more of rejuvenation the appearance of skin surface; improving the appearance of wrinkles and pores; modification of skin conditions associated with ageing, baldness or dyspigmentation; regulation of skin and/or hair pigmentation; modification of skin conditions associated with a skin disease such as atopic dermatitis, psoriasis and depigmmentation (skin and hair) disorder, skin conditions associated UVA irradiation or any other UV induced disease (e.g., apoptosis or inflammation); maintaining skin structural and functional integrity, maintaining skin homeostasis, regulation of skin and/or hair pigmentation and regulation of skin immune function.

In some embodiments of the invention, the compositions (or a formulation comprising thereof) may be utilized for treating eye puffiness, symptoms of aging, protecting the skin, increasing the detoxification of xenobiotics, intervening on pigmentation level, inhibiting melanogenesis, protecting the body against pollution, stimulating the detoxification systems, stimulating self tanning skin activity, stimulating hair and body hair growth, intervening on adipocytes, and promoting lipolysis.

As used herein, the term “treatment” or “prevention”, where used in reference to modifying at least one mechanical and/or physiological condition of the skin, refers to the topical administration/application of an effective amount of a composition of the present invention effective to ameliorate undesired symptoms associated with a skin disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above.

In some embodiments of the invention, the subject may be a healthy subject and the methods utilized according to the invention may be non-therapeutic methods e.g., a cosmetic method. The cosmetic method may also be applicable to a non-healthy subject.

The tem “effective amount” used herein, whether therapeutically or cosmetically effective amount for purposes disclosed herein is determined by such considerations as may be known in the art. In the content of the present disclosure, the amount of the Dead Sea Water in the disclosed compositions must be effective to achieve one or more of the above desired therapeutic or cosmetic effects, depending, inter alia, on the type and severity of the conditions to be modified or the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile, a variety of pharmacological parameters such as half life on the skin, moisturizing level on skin surface, on undesired side effects, if any, on factors such as age and gender, etc.

The compositions of the invention or formulations comprising same may be applied onto the skin by any one method known for application of a standard cream. The application may be for a short period of time, namely the compositions in a suitable form (as disclosed herein) is applied topically and then removed for example within a few minutes. Alternatively, the compositions may be applied and allowed to remain in contact with the skin over longer periods of time. In some embodiments, the composition is allowed to remain on the skin overnight. In some embodiments the composition is allowed to remain of the skin for a period of time of 24 hours, at times for a period of 48 hours. In order to achieve long term effective contact with the skin, the compositions of the invention may be absorbed or loaded onto a carrier which retains its form; such carrier may be a patch, a dressing or a bandage in a form providing sufficient contact with the skin.

For ease of use, the compositions of the invention or formulation comprising same may be formed into a kit or a commercial package and provided along with instructions for use. The compositions comprised in the kit or in the commercial package may be in a quantity and composition suitable for a short term or long term application, for a generic or specific purpose.

In a further one of its aspects the present invention provides compositions according to the invention formulated as a personal skin care product, a dermatological formulation or as a skin care formulation.

In some embodiments the personal skin care product is selected from a cleansing product or a moisturizing product.

In some embodiments the cleansing product is selected from a liquid soap or a bath/shower gel.

In some embodiments the moisturizing product is selected from a cream, a lotion, a gel-cream, a serum, a facial mask, a conditioner or a mask.

In some embodiments, the dermatological formulation is a pharmaceutical or a cosmetic formulation.

Non-limiting examples of formulation which may contain a composition according to the present invention include a lotion, an ointment, a gel, a moisturizer, a sunscreen, a cream, a stick, a spray, an aerosol, foam, a paste, a mousse and a variety of cosmetics or skin-care formulations including solid, semi-solid, or a liquid make-up such as foundations, eye make-up, etc. In some embodiments, the compositions of the invention may be formulated as a lotion, a gel, a cream, a serum or a facial mask.

The formulations according to the invention (cosmetic or therapeutic) may comprise at least one dermatological, cosmetically or pharmaceutically acceptable additive selected amongst inert and effect-inducing additives. Such additive may be introduced to induce or provide additional advantages or characteristics required for particular applications or to endow the formulation with one or more additional physical or chemical attribute.

The at least one additive may be selected, in a non-limiting fashion, from a surfactant, a co-surfactant, a dye, a colorant, a perfume, an optical brightener, a stabilizer, a foam stabilizer, a co-solvent (such as ethanol or isopropyl alcohol, ethylene glycol, isopropylene glycol, 1,3-propane diol, glycerol or water miscible glycol ethers such as ethylene glycol monomethyl ether, diethylene glycol monomethyl ether or polyethylene glycol), a diluent, a preservative, an abrasive, an anti-caking agent, an antistatic agent, a binder, a buffer, a dispersant, an emollient, an emulsifier, a co-emulsifier, a fibrous material, a film forming agent, a UV filter, a fixative, a foaming agent, a foam booster, a gallant, a lubricant, a moisture barrier agent, an opacifier, a plasticizer, a preservative, a propellant, a suspending agent, a thickener, a wetting agent, a liquefier, a rheological agent, a fragrance and a fragrance carrier.

In some embodiments, the at least one additive may be a smoothness enhancer ingredient, such as silica.

In some embodiments, the at least one additive may be a preservative being selected from methylparaben, methyldibromo glutaronitrile, phenethyl alcohol, glyceryl caprilate, propylparaben, methylisothiazolinone, decylene glycol, dehydroacetic acid, phenoxyethanol, ethylhexyl glycerin, potassium sorbate, benzoic acid, 2-methyl-2H-isothiazoline-3-one, polyethylene glycol monococoate, polyethylene glycol dicocoate, polyethylene glycol, iodopropynyl butylcarbamate, 1,2-hexanediol, caprylyl glycol, imidazolidinyl urea, 2,3-bronopol or any combination thereof.

In some embodiments, the additive may be an emollient, being selected from vegetable and animal fats and oils such as castor oil, hydrogenated castor oil, cocoa butter, safflower oil, cottonseed oil, corn oil, olive oil, cod liver oil, almond oil, avocado oil, palm oil, sesame oil, squalene, phytosqalene, kikui oil, chamomilla recutita (matricaria) flower oil, hypericum perforatum oil, soybean oil and vitis vinifera (grape) seed oil; acetoglyceride esters, such as acetylated monoglycerides; alkyl esters of fatty acids having 10 to 24 carbon atoms which include, but are not limited to, methyl, isopropyl, and butyl esters of fatty acids such as hexyl laurate, isohexyl laurate, ethylhexyl palmitate, isohexyl palmitate, isopropyl palmitate, octyl palmitate, decyloleate, isodecyl oleate, hexadecyl stearate decyl stearate, isopropyl isostearate, diisopropyl adipate, diisohexyl adipate, dihexyldecyl adipate, diisopropyl sebacate, lauryl lactate, myristyl lactate, and cetyl lactate; alkenyl esters of fatty acids having 10 to 20 carbon atoms such as oleyl myristate, oleyl stearate, and oleyl oleate; fatty acids having 10 to 20 carbon atoms such as pelargonic, lauric, myristic, palmitic, stearic, isostearic, hydroxystearic, oleic, linoleic, ricinoleic, arachidic, behenic, and erucic acids; fatty alcohols having 10 to 20 carbon atoms such as lauryl, myristyl, cetyl, hexadecyl, stearyl, isostearyl, hydroxystearyl, oleyl, ricinoleyl, behenyl, erucyl, and 2-octyl dodecanyl alcohols; fatty alcohol ethers such as propoxylated fatty alcohols of 10 to 20 carbon atoms which include, but are not limited to, lauryl, cetyl, stearyl, isostearyl, oleyl, and cholesterol alcohols, having attached thereto from 1 to 50 propylene oxide groups; lanolin and lanolin derivatives such as lanolin, lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated sorbitol lanolin, and liquid and semisolid lanolin absorption bases; polyhydric alcohol esters such as ethylene glycol mono and di-fatty acid esters, diethylene glycol mono-and di-fatty acid esters, polyethylene glycol, mono-and di-fatty acid esters, propylene glycol mono-and di-fatty acid esters, polypropylene glycol 2000 monooleate, polypropylene glycol 2000 monostearate, glyceryl mono-and di-fatty acid esters, polyglycerol polyfatty esters, ethoxylated glyceryl monostearate, 1,2-butylene glycol monostearate, 1,2-butylene glycol distearate, polyoxyethylene polyol fatty acid ester, sorbitan fatty acid esters, and polyoxyethylene sorbitan fatty acid esters; Wax esters such as beeswax, spermaceti, myristyl myristate, stearyl stearate; forming a mixture of ether esters; vegetable waxes including, but not limited to, carnauba and candelilla waxes; surface active silicone derivatives such as cyclopentasiloxane, dimethicone, dimethicone crosspolymer, cyclomethicone, cyclomethicone&dimethiconol; caprylic/capric triglyceride; and cholesterol fatty acid esters and or mixtures thereof.

In some embodiments the formulations of the invention may further comprise at least one an effect-inducing agent (active ingredient).

In some embodiments, the effect-inducing agent may be selected from an anti-acne agent, an anti-aging agent, an antibacterial agent, an anti-cellulites agent, an antidandruff agent, an antifungal agent, an anti-inflammatory agent, an anti-irritation agent, an antimicrobial agent, an antioxidant, an antiperspirant agent, an antiseptic agent, a cell stimulant, a cleansing agent, a conditioner, a deodorant, a fragrance ingredient (e.g., perfume, limonene), a depilatory, a detergent, an enzyme, an essential oil, an exfoliant, a fungicide, a glosser, hair conditioner (hair conditioner agent), hair set resin, hair sheen agent, hair waving agent, a humectants, a moisturizer, an ointment base, a perfume, a protein, a skin calming agent, a skin cleanser, a skin conditioner (skin conditioning agent), a skin healing agent, a skin lightening agent, a skin protectant, a skin smoothing agent, a skin softening agent, a skin soothing agent, a sunscreen agent, a UVA and/or UVB filter, a tanning accelerator, vitamins and/or derivatives thereof, a colorant or a flavoring agent.

The cosmetic or pharmaceutical formulations of the invention may also comprise pharmaceutical actives (e.g., a drug), e.g., suitable for topical application, to induce a desired non-systemic effect or at least one systemic effect.

Non-limiting examples of such actives are an antibiotic, an antiviral agent, an analgesic, an antihistamine, an anti-inflammatory agent, an antipruritic, an antipyretic, an anesthetic agent, a diagnostic agent, a hormone, an antifungal agent, an antimicrobial agent, a cutaneous growth enhancer, a pigment modulator, an antiproliferative, an antipsoriatic, a retinoid, an anti-acne medicament, an antineoplastic agent, a phototherapeutic agent, a keratolys and mixtures thereof.

The formulations of the present invention may also comprise at least one active ingredient for skin protection, e.g., anti UVA or UVB agents, sunscreen agents or sun-tanning agents.

As noted above, the compositions of the present invention may be formulated as skin care or dermatological pharmaceutical formulations (including, e.g., toiletries, health and beauty aids and cosmeuticals) used for cosmetic and personal skin-care applications. The term “cosmetic formulation” or “skin care formulation” as used herein relates to a formulations/composition that can be used topically by application to a skin region (without substantially inducing systemic effect) for achieving a cosmetic benefit, hygiene or skin-care or as a basis for delivery of one or more pharmaceutical ingredients. In the context of the present disclosure, in some embodiments the cosmetic formulations are for promoting bodily attractiveness, cover or mask the physical manifestations of a disorder or disease, modulate or alleviate wrinkling, photo-damage, unevenness and dryness in the skin of a subject. The formulations may additionally regulate skin condition and signs of skin aging (all perceptible manifestations as well as any other macro or micro effects) by regulating visible and/or tactile discontinuities in skin texture, including fine lines, wrinkles, enlarged pores, roughness and other skin texture discontinuities associated with aged skin with reduced irritation and dryness.

Unless otherwise defined, all technical and scientific terms used herein have the same meaning as commonly understood by one of ordinary skill in the art to which this invention belongs. Although suitable methods and materials are described below, methods and materials similar or equivalent to those described herein can be used in the practice of the present invention. In case of conflict, the patent specification, including definitions, will control. All materials, methods, and examples are illustrative only and are not intended to be limiting.

The amount or concentration of each of the ingredients of any one of the compositions of the invention may vary. It should be understood that any specific concentration of ingredients provided herein should be taken to mean an approximate concentration. Further, it should be noted that where various embodiments are described by using a given range, the range is given as such merely for convenience and brevity and should not be construed as an inflexible limitation on the scope of the invention. Accordingly, the description of a range should be considered to have specifically disclosed all the possible sub-ranges as well as individual numerical values within that range.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

It is noted that features of certain embodiments of the invention which are described in detail in the context of one aspect of the invention, may be applicable in other aspects of the invention.

### DETAILED DESCRIPTION OF EMBODIMENTS

Before explaining a non-limiting embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Studied Compositions:

In the present disclosure the commercial preparation of Dead Sea minerals known as “Maris Sal” or “Maris Aqua” (Dead Sea Water) was used.

The “Maris Sal” solution has the following composition:

Salt normality (N)

| | |
|---|---|
| Na | 0.118 (2.720 g/l) |
| K | 0.054 (2.100 g/l) |
| Ca | 0.873 (35.000 g/l) |
| Mg | 3.815 (92.700 g/l) |
| Ba | 6.6 × 10⁻⁵ (0.009 g/l) |
| Cd | <1.8 × 10⁻⁷ (<2 × 10⁻⁵ g/l) |
| Co | <3.4 × 10⁻⁵ (<0.002 g/l) |
| Cu | <3.15 × 10⁻⁵ (<0.004 g/l) |
| Cr | <3.85 × 10⁻⁴ (0.02 g/l) |
| Fe | <3.58 × 10⁻⁵ (<0.002 g/l) |
| Li | 5.76 × 10⁻³ (0.040 g/l) |
| Mn | 1.82 × 10⁻⁴ (0.010 g/l) |
| Mo | <1.04 × 10⁻⁶ (<10⁻⁴ g/l) |
| Ni | <3.4 × 10⁻⁵ (<0.002 g/l) |
| Pb | <9.6 × 10⁻⁸ (<2 × 10⁻⁵) |
| Rb | 3.5 × 10⁻⁶ (<3 × 10⁻⁴ g/l) |
| Sb | <1.6 × 10⁻⁷ (<2 × 10⁻⁵ g/l) |
| Sr | 7.6 × 10⁻³ (0.670 g/l) |
| V | <7.9 × 10⁻⁵ (<0.004 g/l) |
| Th | <8.6 × 10⁻⁸ (<2 × 10⁻⁵ g/l) |
| U | <8.4 × 10⁻⁸ (<2 × 10⁻⁵ g/l) |
| Zn | <3.06 × 10⁻⁵ (<0.002 g/l) |
| Cl | 9.75 (346 g/l) |
| Br | 0.175 (14 g/l) |
| B | 0.011 (0.120 g/l) |
| As | 2.7 × 10⁻⁵ (0.002 g/l) |
| I | 6.30 × 10⁻⁷ (8 × 10⁻⁸ g/l) |
| SiO2 | <3.33 × 10⁻⁴ (<0.02 g/l) |
| SiO4 | <2.2 × 10⁻³ (<0.2 g/l) |

Solutions comprising Dead Sea Minerals/Water were prepared by dilutions of the “Maris Sal” solution in demineralized water (DDW). Various concentrations of the “Maris Sal” solution were used.

The various compositions comprising Dead Sea Water, referred to herein as Osmoter compositions (OSM), were used with the following concentrations of Dead Sea Water in demineralized water: 0.25%, 0.5%, 0.8%, 1.0%, 2.0% and 100%.

An extract of Lavender (ArEAUmat Lvanda supplied by CODIF international) has been used for comparison. The Extract has been recently shown to stimulate beta-endorphin production when applied onto the skin [D.Tech. 04-084 GB Areaumats, “beta-endorphins and moisturizing” Laboratories CODIF International].

The compositions have been topically applied onto skin explants as will be detailed herein below. Further, to evaluate the topical effect of the compositions of the invention, for comparison, similar compositions were placed in the studied culture media without topical application thereof (see below).

Further, for the purpose of comparison, a composition comprising Dead Sea Mud has been similarly topically applied on the skin explants samples. The content of the Mud composition used was as follows: 87.33% Dead Sea Silt, 10% Aqua, 1.67% dead sea salt and 1% phenoxyethanol.

Topical Application Assay:

The topical effect of the various compositions on the secretion of beta-endorphin was studied in human skin organ culture model.

Human skin was removed by plastic surgery of the abdomen area (donor age was 38-48 years old). Few hours after skin removal the skin was washed with phosphate buffered saline (PBS) solution and cut into 0.8×0.8 cm (0.16 cm²) explants. The explants were decontaminated for one minute using 70% ethanol wipe. Skin samples were placed in 6 well plate, two skin explants in each well, the well contained 2 ml of culture medium DMEM (Dulbecco's Modified Eagle's Medium, Biological Industries Beit HaEmek, Israel). The skin explants were left for recovery in the medium for 24 hr.

After recovery, 3 μl of the various studied solutions (Osmoter solutions at various DSW concentrations and lavender extract solution) were topically applied onto the skin explants. One sample containing 1% of lavender extract in the culture medium (not topically applied) was used for comparison. Total 12 explants were placed on 6 wells plate—2 for each well with 1 ml of DMEM culture medium. The Experiment was repeated four times for statistical measurements.

After 48 hours the medium was collected from each well and kept frozen at −80° C. for further analysis of beta-endorphin using ELISA immunoassay (see below). The explants were also tested for viability tests using MTT and Resazurin (almar blue) assays (see below). Similar experiments were also conducted at a time interval of 24 hours. Accordingly, after 24 hours the medium was collected from each well and kept frozen at 80° C. for further analysis of beta endorphin and viability.

Beta Endorphin Immunoassay:

The amount of beta-endorphin was determine in culture media by competitive immunoassay, using ELISA high sensitive kit (Peninsula Labratories, BACHEM, LLC, USA, SO#0041195). A polystyrene plate was coated with first antibody specific for a secondary antibody. The secondary antibody was incubated with the tested sample and with a biotinilated beta endorphin and incubated. After washing away unbound beta endorphin (originated from the sample or biotinilated) horseradish peroxide linked the biotinilated beta endorphin, and used to reveal the immobilized complex. A standard solution of human beta endorphin was used as a control.

MTT Assay:

Epidermal viability was evaluated using the MTT colorimetric test of mitochondrial activity. After the explants were incubated for 24 hours or 48 hours, epidermis was separated from dermis by 1-min heating in PBS at 56° C. Samples were transferred into wells containing 150 μl of 0.5 mg/ml (3-4,5-dimethylthiazol-2-yl)-2,5-diphenyl tetrazolium bromide (MTT), (Cat #06896LJ, Sigma, St. Louis, USA) and incubated for 1 hour at 37° C. The resulting precipitated stain (formazan crystals) was extracted for 30 min at room temperature in 0.5 ml isopropanol (Frutarom, Haifa Israel). 100 μl aliquots were transferred into 96-well plates, and optical density was measured in duplicate at 570 nm. The measured values were correlated with epidermal viability.

Resazurin Assay:

Each explant were placed with 300 μl of DMEM containing 0.1 μg/ml Resazurin (Cat #018K3767, Sigma, St. Louis, USA), and incubated for 1 hour at 37° C. the medium solution were transfer to a 96 wells plate and fluorescence was measured using fluoresces reader at 544/590 Ex/Em. The fluorescence was correlated with epidermal viability.

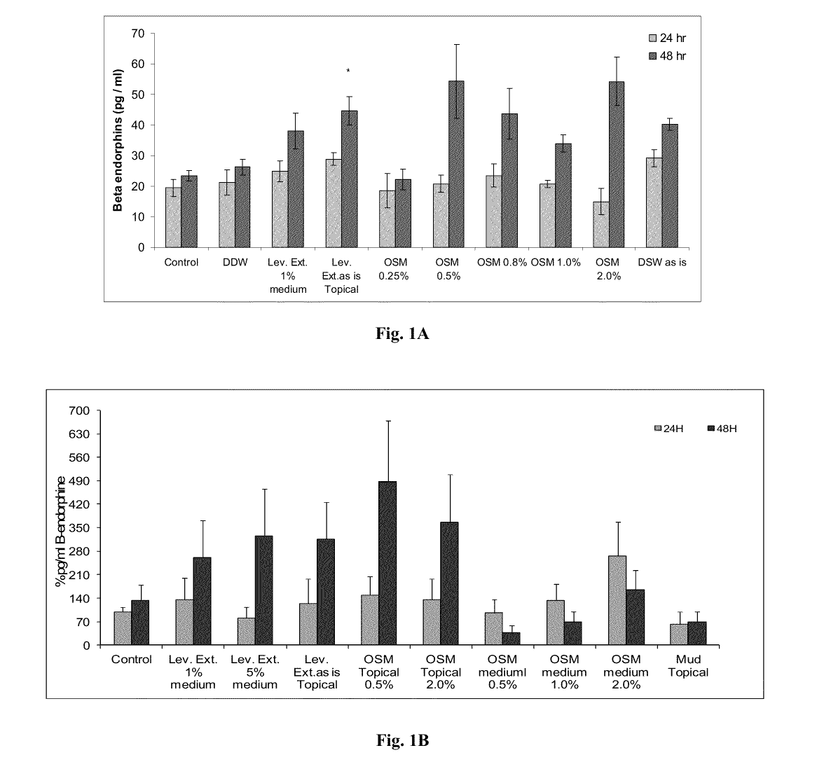

The effect on the secretion of skin beta-endorphins observed with the various compositions which are detailed on Table 1 below is illustrated in FIG. 1A. It is shown in FIG. 1A that beta-endorphin secretion from skin explants is significant when the explants are topically treated with compositions comprising 0.5% DSW and above. The observed effect is predominant with skin explants that were topically treated for a time period of 48 hours.

TABLE 1
Tested samples

| Sample No. | Solution |
|---|---|
| 1 | Control (DMEM) |
| 2 | Control + 3 μl DDW |
| 3 | Lavender extract applied in the medium 1% |
| 4 | Lavender extract topically applied 3 □ μl |
| 5 | Osmorer 0.25% 3 μl topical applied |
| 6 | Osmorer 0.5% 3 μl topical applied |
| 7 | Osmorer 0.8% 3 μl topical applied |
| 8 | Osmorer 1.0% 3 μl topical applied |
| 9 | Osmorer 2.0% 3 μl topical applied |
| 10 | Osmorer as is (100%) 3 μl topical applied |

FIG. 1B illustrates the effect of the compositions according to the invention on skin beta-endorphin secretion upon topical application thereof onto skin explants. While no effect of beta-endorphin secretion was observed when Dead Sea Water were only present in the aforementioned assay medium (the tested samples are designated in the Figure as “OSM medium 0.5%”, “OSM medium 1.0%” and “OSM medium 2.0%”), a significant effect was observed when the compositions were topically applied on the skin explants (the tested samples are designated in the Figure as “OSM Topical 0.5%” and “OSM topical 2.0%”). FIG. 1B also illustrates the significant effects of the compositions comprising DSW compared to Dead Sea Mud (the latter sample is designated in the figure as “Mud Topical”). While no beta-endorphin secretion was observed in the skin explants which were treated with Dead Sea Mud, a significant effect was observed with explants topically treated with compositions comprising 0.5% and 2.0% Dead Sea Water. It is noted that the beta endorphins content in FIG. 1B is provided in % pg/ml i.e., it is normalized as a percentage compared to a control (no treatment) expressed as 100%.

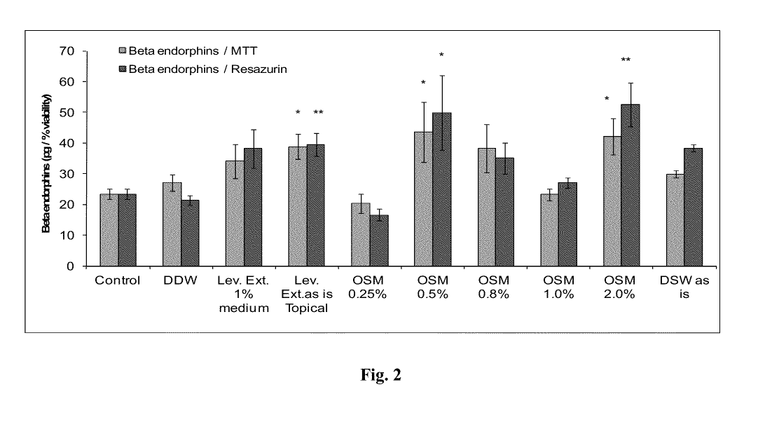

FIG. 2 illustrates the effect on the secretion of skin beta-endorphins observed 48 hours following topical application of the various compositions which are detailed on Table 1. The data is normalized taking into consideration viability measurements conducted with MTT and Resazuin assays. The corresponding data is summarized in Table 2 below.

TABLE 2
Viability test results after treatment of skin samples for 48 hours

| | | Viability Tests | |
|---|---|---|---|
| | Beta Endorphin | MTT | Resazurin |
| Treated Sample | (pg/ml) | (% of viability) | (% of viability) |
| Control | 23 ± 2 | 100 ± 2 | 100 ± 5 |
| DDW | 26 ± 3 | 97 ± 3 | 123 ± 18 |
| Lav. Ext. Med 1% | 38 ± 6 | 119 ± 5 | 137 ± 15 |
| Lav Ext. Topical | 45 ± 5 | 111 ± 5 | 115 ± 8 |
| OSM 0.25% | 22 ± 3 | 109 ± 13 | 134 ± 11 |
| OSM 0.5% | 54 ± 12 | 128 ± 5 | 138 ± 11 |
| OSM 0.8% | 44 ± 8 | 137 ± 6 | 124 ± 7 |
| OSM 1.0% | 34 ± 3 | 146 ± 16 | 125 ± 11 |
| OSM 2.0% | 54 ± 8 | 105 ± 9 | 85 ± 5 |
| OSM 100% | 40 ± 2 | 135 ± 5 | 105 ± 8 |

1. A method of inducing secretion of beta-endorphin in a skin region of a subject, the method comprising topically applying a composition comprising at least 0.5% Dead Sea Water onto said skin region of said subject.

2. The method according to claim 1, wherein the secretion is induced over a period of 48 hours post the topical application of said composition onto said skin region of the subject.

3. The method according to claim 1, wherein said Dead Sea Water constitutes 0.5%, 0.75%, 0.8%, 1.0% or 2.0% of the total weight of said composition.

4. The method according to claim 1, wherein said Dead Sea Water constitutes 0.5% of the total weight of said composition.

5. The method according to claim 4, wherein said composition is a rinse off composition.

6. The method according to claim 1, wherein said Dead Sea Water constitutes 2.4% of the total weight of the composition.

7. The method according to claim 6, wherein said composition is a leave-on composition.

8. The method according to claim 1, wherein said skin region is at least one facial skin region.

9. A method of inducing secretion of beta-endorphin in a skin region of a subject, the method comprising topically applying a composition comprising at least 0.5% Dead Sea Water onto said skin region of said subject, wherein the secretion is induced over a period of 48 hours post the topical application of said composition onto said skin region of the subject.

10. A method of modifying at least one condition of a skin region of a subject, the method comprising topically applying a composition comprising at least 0.5% Dead Sea Water onto the skin region of the subject, wherein the at least one condition is selected from the group consisting of mechanical and physiological skin condition and wherein the modifying of the at least one skin condition is associated with induction of skin beta-endorphin secretion in the skin region.

11. The method according to claim 10, wherein said secretion is induced over a period of 48 hours post topical application of said composition onto said skin region of said subject.

12. The method according to claim 10, wherein said modifying at least one skin condition is associated with protecting and/or improving and/or rejuvenating the state of said skin, preventing and/or treating imperfections of said skin of said subject and/or treating or preventing at least one disease or disorder of said skin.

13. The method according to claim 10, wherein said Dead Sea Water constitutes 0.5%, 0.75%, 0.8%, 1.0% or 2.0% of the total weight of said composition.

14. The method according to claim 10, wherein said Dead Sea Water constitutes 0.5% of the total weight of said composition.

15. The method according to claim 14, wherein said composition is a rinse off composition.

16. The method according to claim 10, wherein said Dead Sea Water constitutes 2.4% of the total weight of the composition.

17. The method according to claim 16, wherein said composition is a leave-on composition.

18. The method according to claim 10, wherein said skin region is at least one facial skin region.

19. The method according to claim 10, wherein said composition further comprises at least one additive.

20. The method according to claim 19, wherein said additive is a drug molecule.
